SEQUENCE LISTING (6 sequences)

SEQ ID NO: 1 - DNA, 70 bp, Artificial
  synthetic oligonucleotide sequence
ctggtgacaactaaacgttgccacctgcgttccattaattacgaaacatcctgccagagccgacgccagt

SEQ ID NO: 2 - DNA, 1085 bp, Artificial
  thyA gene from L. Lactis under control of constitutive promoter
gcggccgctctaaaaaatgccctcttgggttatcaagagggtcattatatttcgcggaattcatgctatcgacgtctgatatcagagctctgttgacaattaatcatcggcatagtatatcggcatagtataatacgacaaggtgaggaactaaaccatgacttacgcagacaagatttttaaacaaaatatccaaaatatccttgataacggtgttttttcagaaaatgcacgtcccaagtataaggatggtcaaaccgcaaatagtaagtatgtaacaggctctttcgttacttatgatttgcaaaaaggtgagtttccaattaccactttgcgtccaattccaattaaatcggcaattaaagaattgatgtggatttatcaagatcaaacaagtgaacttgctattcttgaagaaaaatatggagtcaaatactggggcgaatggggaattggcgacggtacgattggacaacgctatggggcaacagtcaaaaaatataatattattggaaaattattggatggtttagcaaaaaatccttggaatcgccgtaatatcattaatctttggcaatatgaagattttgaggaaactgaaggacttttgccttgtgctttccaaacaatgtttgatgttcgccgtgaacaagatggtcagatttacttggatgcgactcttattcaacgttcaaatgatatgcttgttgcgcatcatatcaatgccatgcaatatgttgctttacaaatgatgattgcaaaacatttttcttggaaagttggaaaatttttctatttcgtaaataatttgcatatttatgataatcagtttgaacaggcaaatgagttagttaagcgaacagcttctgacaaggagccccgtttggtgcttaatgttcctgacggaacaaactttttcgatattaaaccagaagattttgagcttgtggactatgaaccggtaaaacctcaattaaaatttgatttggcaatttaggcgcgcctgtaatcacactggctcaccttcgggtgggcctttctgctttaaaaaaaacgggccggcgcgaacgccggcccgcggccgc

SEQ ID NO: 3 - DNA, 69 bp, Artificial
  Forward PCR primer
accatgattacggattcactggccgtcgttttacaacgtcgtgactgctgatccactagttctagagcg

SEQ ID NO: 4 - DNA, 69 bp, Artificial
  reverse PCR primer
tttttgacaccagaccaactggtaatggtagcgaccggcgctcagcctaaagggaacaaaagctgggtg

SEQ ID NO: 5 - DNA, 796 bp, Artificial
  thyA removal sequence
gccggcggtcatcagatgcgttttaacctgcaagatggattcccgctggtgacaactaaacgttgccacctgcgttccatcatccatgaactgctgtggtttctgcagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaattacgaaacatcctgccagagccgacgccagtgtgcgtcggtttttttaccctccgttaaattcttcgagacgccttccgccggc

SEQ ID NO: 6 - DNA, 3617 bp, Artificial
  lambda removal sequence
gccggcagggtaatcggggaaggattccacgctgcagcacctggtgtcagggatgcaaaatagtgttgagcatcgaaattctgcgcttcttttgccgacagaatcgggcgagaagaggtaccaggcgcggtttgatcagaaggacgttgatcgggcggggttgagctacaggcggtcagcgtcacgccaaaagccaatgccagcgccagacgggaaactgaaaatgtgttcacaggttgctccgggctatgaaatagaaaaatgaatccgttgaagcctgcttttttatactaacttgagcgaaacgggaaggtaaaaagacaaaaagttgtttttaatacctttaagtgataccagatggcattgcgccatctggcagagtgattaactaaacatcgcagtaatcgaggcgcttgccagagagtggaaatgaacgttaaacccgaccatcgcgccgctggcaccttcatcgacatcaatacgttctatatccagcgcgtgaacggtaaaaatgtagcgatgagtttcgcctttcggcggtgctgcgccatcgtacccggttttaccaaagtcggtacgcgtctgcaaaacgccgtctggcattgctaccagaccagagccaaacccttgcggtaatacgcgggtatcagcgggtaagttaacaactacccagtgccaccagccggagccggttggcgcatccgggtcgtagcaggtgacaacaaaacttttcgttcccgcaggaacatcatcccacgccagatgcggtgaaatattatcgccatcgtaacccatgccgttaaagacatgacgatgcggcaatttatcgccatcgcgcagatcgttactgatgagtttcatgaaccctcctttcttgtttgcagaaagtgtagccagaaaccctcacgcggacttctcgttattggcaaaaaaatgtttcatcctgtaccgcgcggttaaccgctgcggtcagacgctgcaactgttgcgggagaataatatagggcggcatcaggtaaatcagtttgccaaaaggccggatccagacaccctgttcgacaaagaatttttgcagcgccgccatattcaccggatgagtggtttcgaccacgccaatggcccccagtacgcgcacatcggcaaccatttcggcatcacgggcgggggcaagttgctcgcgcagctgtacttcaatatccgccacctgttgctgccagtcgccagattcgagaatcgccaggctggcgtttgctgccgcgcaggccagcggattgcccataaaagttggcccatgcataaagcaaccggcttcaccgttactgatggtttctgcaacctcgcgcgtggtgagtgtggcggaaagggtcattgtgccgccggttaaggctttaccgaggcacaaaatgtccggcgcgatttctgcatgttcacaggcaaacagtttcccggtacgaccaaatccagtggcgatctcgtcggcaatcagcaagataccttcgcgatcgcatattttgcggattcgttttaaccattccggatggtacatgcgcatcccgcctgcgccctggacaatcggctcaatgatcaccgccgcgatttcatgacgatgcgccgccatcaggcgggcaaagcccaccatatcgcgctcatcccattcgccatccatgcggctttgcggggcgggagcaaacaggttttctggcaggtagcctttccacagactgtgcattgagttatccggatcgcacaccgacatcgcgccaaaggtatcgccatgataaccattgcggaaggtcagaaaacgctggcgcgcttcgcctttggcttgccagtactgcaacgccattttcatcgccacttccaccgctacggaaccggagtccgcgagaaaaacgcactccagcggttgcggcgtcatcgccaccagtttgcggcacagctcaatggctggcgcatgggtgataccgccaaacatcacatgcgacatggcatcaatttgcgacttcatcgccgcattaagctgcgggtgattgtagccgtggatcgccgcccaccaggacgacataccgtcaaccaggcgtctgccgtcagacaaaatcagctcgcaaccttcggcgctcaccaccggataaaccggcagaggggaggtcatggatgtgtatgggtgccagatatggcgttggtcaaaggcaagatcgtccgttgtcataatcgacttgtaaaccaaattgaaaagatttaggtttacaagtctacaccgaattaacaacaaaaaacacgttttggagaagccccatggctcaccgcccacgctggacattgtcgcaagtcacagaattatttgaaaaaccgttgctggatctgctgtttgaagcgcagcaggtgcatcgccagcatttcgatcctcgtcaggtgcaggtcagcacgttgctgtcgattaagaccggagcttgtccggaagattgcaaatactgcccgcaaagctcgcgctacaaaaccgggctggaagccgagcggttgatggaagttgaacaggtgctggagtcggcgcgcaaagcgaaagcggcaggatcgacgcgcttctgtatgggcgcggcgtggaagaatccccacgaacgcgatatgccgtacctggaacaaatggtgcagggggtaaaagcgatggggctggaggcgtgtatgacgctgggcacgttgagtgaatctcaggcgcagcgcctcgcgaacgccgggctggattactacaaccacaacctggacacctcgccggagttttacggcaatatcatcaccacacgcacttatcaggaacgcctcgatacgctggaaaaagtgcgcgatgccgggatcaaagtctgttctggcggcattgtgggcttaggcgaaacggtaaaagatcgcgccggattattgctgcaactggcaaacctgccgacgccgccggaaagcgtgccaatcaacatgctggtgaaggtgaaaggcacgccgcttgccgataacgatgatgtcgatgcctttgattttattcgcaccattgcggtcgcgcggatcatgatgccaacctcttacgtgcgcctttctgccggacgcgagcagatgaacgaacagactcaggcgatgtgctttatggcaggcgcaaactcgattttctacggttgcaaactgctgaccacgccgaatccggaagaagataaagacctgcaactgttccgcaaactggggctaaatccgcagcaaactgccgtgctggcaggggataacgaacaacagcaacgtcttgaacaggcgctgatgaccccggacaccgacgaatattacaacgcggcagcattatgagctggcaggagaaaatcaacgcggcgctcgatgcgcggcgtgctgccgatgccctgcgtcgccgttatccggtggcgcaaggagccggacgctggctggtggcggatgatcgccagtatctgaacttttccagtaacgattatttaggtttaagccatcatccgcaaattatccgtgcctggcagcagggggcggagcaatttggcatcggtagcggcggctccggtcacgtcagcggttatagcgtggtgcatcaggcactggaagaagagctggccggc

(19) United States
(12) Patent Application Publication
(10) Pub. No.: US 2007/0243616 A1
(43) Pub. Date: Oct. 18, 2007

(54) IN VIVO ALTERATION OF CELLULAR DNA

(75) Inventors: George M. Church, Brookline, MA (US); Nikos Reppas, Brookline, MA (US)

Correspondence Address: BANNER & WITCOFF, LTD., 28 STATE STREET, 28th FLOOR, BOSTON, MA, 02109-9601, US

(73) Assignee: President and Fellows of Harvard College, Cambridge, MA (US)

(21) Appl. No.: 11/749,489

(22) Filed: May 16, 2007

Related U.S. Application Data
(63) Continuation of application No. PCT/US05/41953, filed on Nov. 16, 2005.
(60) Provisional application No. 60/628,211, filed on Nov. 16, 2004.

(51) Int. Cl. C12N 15/87; C12N 15/00

(52) U.S. Cl. 435/463; 435/440; 435/471

(57) ABSTRACT

The present invention relates to methods, cells and compositions for performing in vivo recombination without the need for clonal selection. These methods, cells and compositions are useful for the automation of recombination. The present invention provides methods for performing sequential steps in genome engineering, as well as for achieving parallel and multiplex genome engineering.

### RELATED U.S. APPLICATIONS

This application claims priority from PCT Application Number PCT/US2005/041953, filed Nov. 16, 2005; which claims priority from U.S. Provisional Patent Application Ser. No. 60/628,211, filed on Nov. 16, 2004, both of which are hereby incorporated herein by reference in their entirety for all purposes.

### STATEMENT OF GOVERNMENT INTERESTS

This invention was made with government support under the U.S. Department of Energy GTL grant number DE-FG02-02ER63445. The Government has certain rights in the invention.

### FIELD OF THE INVENTION

The present invention relates to novel methods of in vivo, reciprocal DNA exchange (i.e., recombination) in a target cell.

### BACKGROUND OF THE INVENTION

Homologous recombination is the method by which portions of DNA between homologous DNA sequences are exchanged in a reciprocal fashion. During homologous recombination, two duplex DNA molecules are broken and DNA strands are exchanged. Homologous recombination systems have been designed in order to enable the production of mutations in the DNA of a target organism (see Link et al. (1997) J. Bacteriol. 79:6228; Storici et al. (2001) Nat. Biotechnol. 19:773; Kolisnychenko et al. (2002) Genome Res. 12:640). One recombination system that has been recently developed is the λ system in E. coli (see Yu et al. (2000) Proc. Natl. Acad. Sci. U.S.A. 97:5978; and Ellis et al. (2001) Proc. Natl. Acad. Sci. U.S.A. 98:6742). In this system, a modified λ prophage is used to recombine an exogenous linear DNA sequence with a DNA sequence present in a bacterial cell. A temperature-dependent repressor tightly controls prophage expression, and recombination can be stimulated by shifting the temperature of the bacterial cell to 42° C.

Current methods of homologous recombination require multiple steps such as isolating colonies, screening colonies, growing colonies, preparing colonies for subsequent rounds of DNA uptake and the like. These steps are both costly and time consuming. Accordingly, there is a need for an improved system of recombination that reduces the number of steps necessary to achieve desired recombinant organisms.

### SUMMARY OF THE INVENTION

The present invention is based on the discovery of a method of in vivo, reciprocal DNA exchange (i.e., recombination) in a target cell and selection that reduces the need for multiple steps such as isolating colonies, screening colonies, growing colonies, preparing colonies for subsequent rounds of DNA uptake and the like.

Embodiments of the present invention are directed to methods of serially altering one or more nucleic acid sequences of interest (e.g., endogenous genes) in a target cell. In one aspect, methods of serially altering one or more nucleic acid sequences of interest are performed in liquid media without the need for screening using solid media. Such serial alteration in liquid media can be performed in a single apparatus such as a microfuge tube, test tube, a cuvette, a microscope slide, a multi-well plate, a microfiber, a flow system (e.g., microfluidics) or the like. In one aspect, methods of serially altering one or more nucleic acid sequences of interest in a target cell include the addition of one or more nucleic acids into the target cell. In another aspect, methods of serially altering one or more nucleic acid sequences of interest in a target cell include the removal of one or more nucleic acid sequences of interest from the target cell. In another aspect, methods of serially altering one or more nucleic acid sequences of interest in a target cell include the removal or alteration of one or more endogenous genes, intergenic nucleic acid sequences, and/or non-geneic artificial DNA in the target cell.

According to certain embodiments of the present invention, a first exogenous nucleic acid sequence having at least one positive selection marker and having at least one negative selection marker is introduced into a target cell that expresses an inducible recombination system. The recombination system is induced and a positive selection for a recombinant target cell is performed. A second exogenous nucleic acid sequence is then introduced into the recombinant target cell to remove the positive and negative selection markers. The recombination system is induced and a negative selection for recombinant cells in which the markers have been removed is performed to generate a target cell that contains the desired nucleic acid addition(s) or deletion(s) in one or more nucleic acid sequences of interest. In certain aspects, the present invention is directed to cells made by the methods described herein. In other aspects, the present invention is directed to compositions used in the methods described herein.

In one embodiment, the present invention provides a method of in vivo serial alteration of a nucleic acid sequence of interest in a target cell. The method may include the steps of providing target cells in an apparatus, contacting the target cells in the apparatus with a first exogenous nucleic acid sequence having a selection marker, allowing nucleic acid exchange to occur, and contacting the target cells in the apparatus with a first selection substrate, in which cells that survive express the selection marker, removing the first selection substrate from the target cells in the apparatus, contacting the target cells in the apparatus with a second exogenous nucleic acid sequence, allowing nucleic acid exchange to occur, and contacting the target cells in the apparatus with a second selection substrate, in which cells that die express the selection marker, and in which a cell that survives comprises an in vivo serial alteration of a nucleic acid sequence of interest.

Embodiments of the present invention have particular application in automating in vivo, reciprocal DNA exchange (i.e., recombination) in a target cell. In particular, the present invention enables the use of microfluidics for introducing DNA into target cells and for selecting target cells having undergone successful DNA rearrangement. Accordingly, the present invention is useful for massively parallel DNA rearrangement as well as multiplex DNA rearrangement.

Embodiments of the present invention also have particular application in global alterations in the genome of a target cell and/or target organism. In certain aspects, the methods, cells and compositions described herein can be used to create a target cell and/or target organism having novel amino acids or codons. In other aspects, the methods, cells and compositions described herein can be used to remove restriction nuclease sites from a target cell and/or target organism.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 depicts a schematic of directed mutagenesis of the E. coli genome using λ Red. The λ recombination proteins (Exo, Bet, Gam) are induced by a 42° C., ten minute heat shock. ‘+’ and ‘−’ indicate positive and negative selection markers, respectively. For every locus modified, a pair of diagnostic PCR primers were designed such that wild-type, cassette-integrated and mutant versions could be determined by size alone.

FIG. 2 depicts a schematic of the construction of MG1655λ-ΔthyA by λ Red-mediated integration of an oligonucleotide designed to remove 4/5 of the gene. thyA could not be deleted in its entirety because it contains at its 3′ end an essential terminator sequence associated with the gene upstream (Bell-Pedersen et al. (1991) J. Bacteriol. 173:1193). Thymidine was present at 100 μg/mL and trimethoprim was present at 4 μg/mL.

FIG. 3 depicts a schematic of the construction of the pThyA PCR template. The thyA (L. lactis) gene is under the control of the synthetic constitutively active promoter P_{EM7}, the whole unit being bounded by bidirectional transcription terminators (black bowties).

FIGS. 4A-4C depict schematics of thyA-mediated mutagenesis. (A) depicts a thyA-mediated deletion of a non-essential coding sequence (lacZ). Both selections were performed in minimal medium. (B) depicts a thyA-mediated point mutation of nonessential coding sequence (thrA, trpC, tyrA, met. In this case, the original sequence was regenerated, but one could use the same approach to incorporate a base pair change, e.g., in the middle of the targeting regions of the oligonucleotide. (C) depicts a thyA-mediated point mutation of the essential coding sequence (serS). In this case, the point mutation was silent and designed to generate an NsiI site that could be easily detected by restriction digestion of the appropriate PCR product. A double stranded PCR product was used to evict the thyA cassette instead of an oligonucleotide. Depending on which side of the T mutation the left-hand recombination occurs, the mutation either will or will not be fixed into the essential gene.

FIG. 5 depicts a schematic of positive and negative selection on solid media. Step II: 2 mL log phase cells per electroporation, approximately 25 ng thyA PCR cassette leading to 4×10³-1×10⁵ thyA+ integrants. Step III: 0.5 mL log phase cells per electroporation, 100 ng oligonucleotide leading to 1×10⁴-4×10⁵ ΔthyA cells.

FIG. 6 depicts a schematic of positive and negative selection in liquid media. One benefit of such an approach is that the clonal bottleneck characteristic of solid media selection (such as in FIG. 5) is not present. Two rounds of liquid amplification were performed at each step. Using this method with lacZ mutagenesis (such as in FIG. 4A), 84/84 ΔlacZ mutants were obtained on the final non-selective plate.

FIGS. 7A-7B depict data obtained using Affymetrix arrays. R was plotted versus genomic probe position. (A) depicts the analysis of MG1655λ-galK::cat versus MG1655 genomic DNA using Affymetrix E. coli arrays. R=log [MG1655λ-galK::cat/MG1655] intensity ratio for any given probe whose corresponding position in the genome is plotted on the x-axis. A sharp negative deviation in such a graph was indicative of a deletion in MG1655λ-galK::cat relative to MG1655. The one deviation is examined further in (B). (B) depicts a close-up of the 0.8 Mb region in (A). The only two deletions present were those that were expected.

### DETAILED DESCRIPTION OF THE INVENTION

The present invention provides novel methods for performing reciprocal DNA exchange (i.e., recombination) combined with selection to achieve modification of one or more nucleic acid sequences of interest (i.e., target nucleic acids). Embodiments of the present invention are based on the discovery that reciprocal DNA exchange (i.e., recombination) can be performed using a positive/negative selection system in the absence of clonal selection. Surprisingly, a recombination efficiency of nearly 100% has been achieved using the methods, cells and compositions described herein.

According to one embodiment of the present invention, a target cell having an inducible recombination system is provided. A first exogenous nucleic acid sequence having at least one positive selection marker and having at least one negative selection marker is introduced into a target cell that expresses an inducible recombination system. The recombination system is induced and a positive selection for a recombinant target cell is performed. A second exogenous nucleic acid sequence is then introduced into the recombinant target cell to remove the positive and negative selection markers. The recombination system is induced and a negative selection for recombinant cells in which the markers have been removed is performed to generate a target cell that contains the desired nucleic acid addition(s) or deletion(s). In certain embodiments, the positive and negative selection marker is the same marker (e.g., thyA).

In certain aspects, the positive selection marker is a gene that allows growth in the absence of an essential nutrient, such as an amino acid. For example, in the absence of thymine and thymidine, cells expressing the thyA gene survive, while cells not expressing this gene do not. A variety of suitable positive/negative selection pairs are available in the art. For example, various amino acid analogs known in the art could be used as a negative selection, while growth on minimal media (relative to the amino acid analog) could be used as a positive selection.

Visually detectable markers are also suitable for use in the present invention, and may be positively and negatively selected and/or screened using technologies such as fluorescence activated cell sorting (FACS) or microfluidics. Examples of detectable markers include various enzymes, prosthetic groups, fluorescent markers, luminescent markers, bioluminescent markers, and the like. Examples of suitable fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of suitable bioluminescent markers include, but are not limited to, luciferase (e.g., bacterial, firefly, click beetle and the like), luciferin, aequorin and the like. Examples of suitable enzyme systems having visually detectable signals include, but are not limited to, galactosidases, glucorinidases, phosphatases, peroxidases, cholinesterases and the like.

In other aspects, the positive selection marker is a gene that confers resistance to a compound which would be lethal to the cell in the absence of the gene. For example, a cell expressing an antibiotic resistance gene would survive in the presence of an antibiotic, while a cell lacking the gene would not. For instance, the presence of a tetracycline resistance gene could be positively selected for in the presence of tetracycline, and negatively selected against in the presence of fusaric acid. Suitable antibiotic resistance genes include, but are not limited to, genes such as ampicillin-resistance gene, neomycin-resistance gene, blasticidin-resistance gene, hygromycin-resistance gene, puromycin-resistance gene, chloramphenicol-resistance gene and the like.

In certain aspects, the negative selection marker is a gene that is lethal to the target cell in the presence of a particular substrate. For example, the thyA gene is lethal in the presence of trimethoprim. Accordingly, cells that grow in the presence trimethoprim do not express the thyA gene. Negative selection markers include, but are not limited to, genes such as thyA, sacB, gnd, gapC, zwf, talA, talB, ppc, gdhA, pgi, fbp, pykA, cit, acs, edd, icdA, groEL, secA and the like.

The exogenous nucleic acid can be targeted for delivery to target prokaryotic or eukaryotic cells via conventional transformation or transfection techniques. As used herein, the terms “transformation” and “transfection” are intended to refer to a variety of art-recognized techniques for introducing an exogenous nucleic acid sequence (e.g., DNA) into a target cell, including calcium phosphate or calcium chloride co-precipitation, DEAE-dextran-mediated transfection, lipofection, electroporation, optoporation, injection and the like. Suitable methods for transforming or transfecting target cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A target cell can be any prokaryotic or eukaryotic cell. For example, target cells can be bacterial cells such as E. coli cells, insect cells such as Drosophila melanogaster cells, plant cells, yeast cells, amphibian cells such as Xenopus laevis cells, nematode cells such as Caenorhabditis elegans cells, or mammalian cells (such as Chinese hamster ovary cells (CHO), mouse cells, African green monkey kidney cells (COS), fetal human cells (293T) or other human cells). Other suitable target cells are known to those skilled in the art. Both cultured and explanted cells may be used according to the invention. The present invention is also adaptable for in vivo use using viral vectors including, but not limited to, replication defective retroviruses, adenoviruses, adeno-associated viruses and the like.

Target cells useful in the present invention include human cells including, but not limited to, embryonic cells, fetal cells, and adult stem cells. Human stem cells may be obtained, for example, from a variety of sources including embryos obtained through in vitro fertilization, from umbilical cord blood, from bone marrow and the like. In one aspect of the invention, target human cells are useful as donor-compatible cells for transplantation, e.g., via alteration of surface antigens of non-compatible third-party donor cells, or through the correction of genetic defect in cells obtained from the intended recipient patient. In another aspect of the invention, target human cells are useful for the production of therapeutic proteins, peptides, antibodies and the like.

The target cells of the invention can also be used to produce nonhuman transgenic, knockout or other genetically-modified animals. Such animals include those in which a gene or nucleic acid is altered in part, e.g., by base substitutions and/or small or large insertions and/or deletions of target nucleic acid sequences. For example, in one embodiment, a target cell of the invention is a fertilized oocyte or an embryonic stem cell into which the addition or deletion of one or more nucleic acids has been performed. Such target cells can then be used to create non-human transgenic animals in which exogenous detectable translation product sequences have been introduced into their genome. As used herein, a “transgenic animal” is a non-human animal, such as a mammal, e.g., a rodent such as a ferret, guinea pig, rat, mouse or the like, or a lagomorph such as a rabbit, in which one or more of the cells of the animal includes a transgene. Other examples of transgenic animals include non-human primates, cows, goats, sheep, pigs, dogs, cats, chickens, amphibians, and the like. A transgene is exogenous DNA that is integrated into the genome of a cell from which a transgenic animal develops and which remains in the genome of the mature animal. A knockout is the removal of endogenous DNA from a cell from which a knockout animal develops, which remains deleted from the genome of the mature animal. Methods for generating transgenic and knockout animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al., and in Hogan, B., Manipulating the Mouse Embryo, (Cold Spring Harbor Laboratory Press, Cold Spring Harbor, N.Y., 1986).

This invention is further illustrated by the following examples, which should not be construed as limiting. The contents of all references, patents and published patent applications cited throughout this application are hereby incorporated by reference in their entirety for all purposes.

### EXAMPLE I

### Recombination Systems

Many recombination systems are useful in the present invention. Suitable recombination systems include, but are not limited to: 1) linear homologous recombination using two crossover sites near the ends of the sequence of interest (e.g., λ Red); 2) circle homologous integration followed by a second resolving recombination; 3) linear, sequence-specific recombination (e.g., via a phage integrase such as λ or phiC31); and 4) sequence-specific circle integration. Linear recombination systems may be used with one positive/negative selection marker. Circle recombination systems may be used with two positive/negative selection markers, one to replace each end. For example, an integrant into the DNA sequence ABCD could have the general structure: AB-attL-positive/negative(L)-A-new-CD-positive/negative(R)-attR-CD, wherein attL and attR are the flanking homology regions (in the case of circle homologous integration) or integrase att sites (in the case of sequence-specific circle integration). For sequence-specific recombination systems, useful integrases include, but are not limited to, integrases that are known in the art, designed or selected amino acid sequences that aid fidelity or efficiency in targeting beyond simple homology, or combinations thereof.

### EXAMPLE II

### Genome Engineering Using λ Red

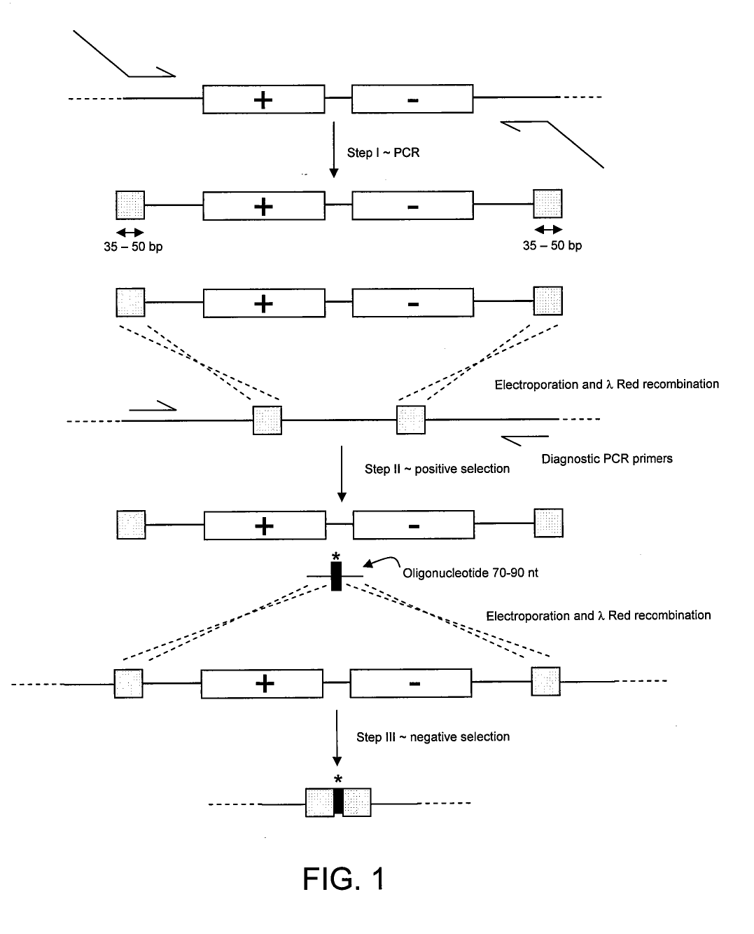

The general scheme exploits inducible λ “Red” linear recombination, which is known in the art (Yu et al., supra; and Ellis et al. (2001) Proc. Natl. Acad. Sci. U.S.A. 98:6742; incorporated herein by reference in their entirety for all purposes) and depicted in FIG. 1.

Three E. coli metabolic genes have been reported to have particularly specific positive and negative selections: pyrF, thyA, and proA. Positive and negative selection of these three genes are tabulated in Table 1. It was determined that the strain background, E. coli MG1655 with the λ Red prophage integrated at the bio locus (MG1655%), had an exceedingly high intrinsic resistance to 5-FOA.

TABLE 1

| Gene | Positive Selection | Negative Selection |
|---|---|---|
| pyrF | defined medium | defined medium with uracil containing |
| | lacking uracil | 5-fluoroorotic acid |
| thyA | defined medium | defined medium with thymidine |
| | lacking thymidine | containing trimethoprim |
| proA | defined medium | defined medium with proline containing |
| | lacking proline | 4-nitropyridine-1-oxide |

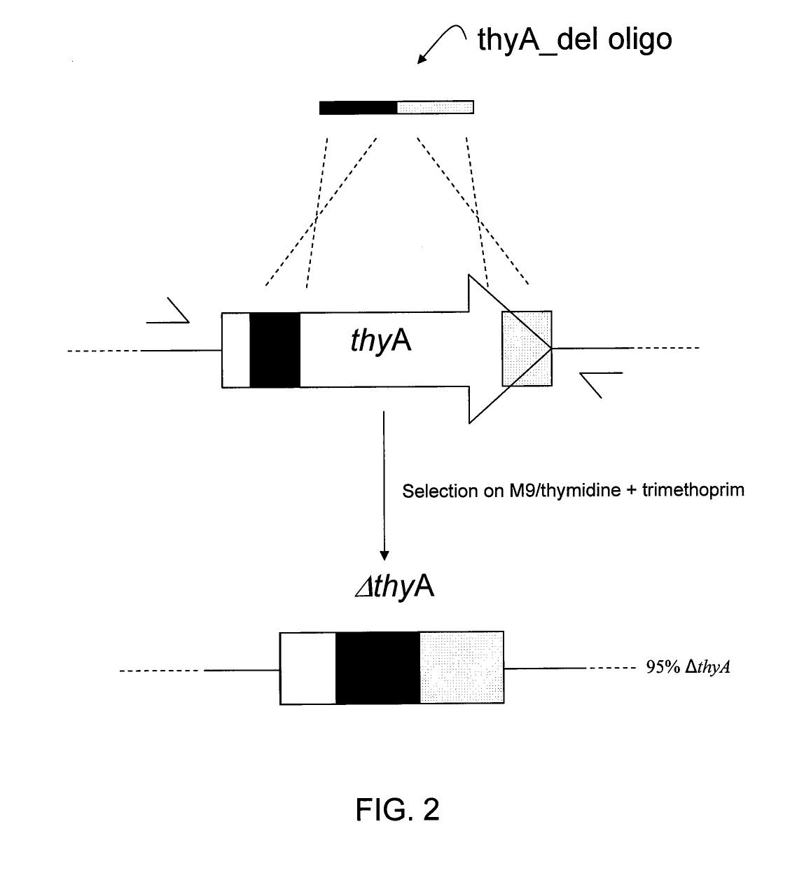

In contrast, thyA, which encodes thymidylate synthase, was observed to work well in both positive and negative selection modes. Upon λ Red recombination of the thyA_del oligonucleotide with the endogenous thyA gene, followed by selection on thymidine+trimethoprim minimal medium, 95% of the clones were ΔthyA as determined by diagnostic PCR (set forth in FIG. 2). This procedure was used to generate the MG1655λ-ΔthyA strain to be used as the background for chromosomal engineering as described further herein. The MG1655λ-ΔthyA strain is auxotrophic for thymine/thymidine, stably trimethoprim-resistant, and grows (in the presence of thymine/thymidine) at the wild-type growth rate.

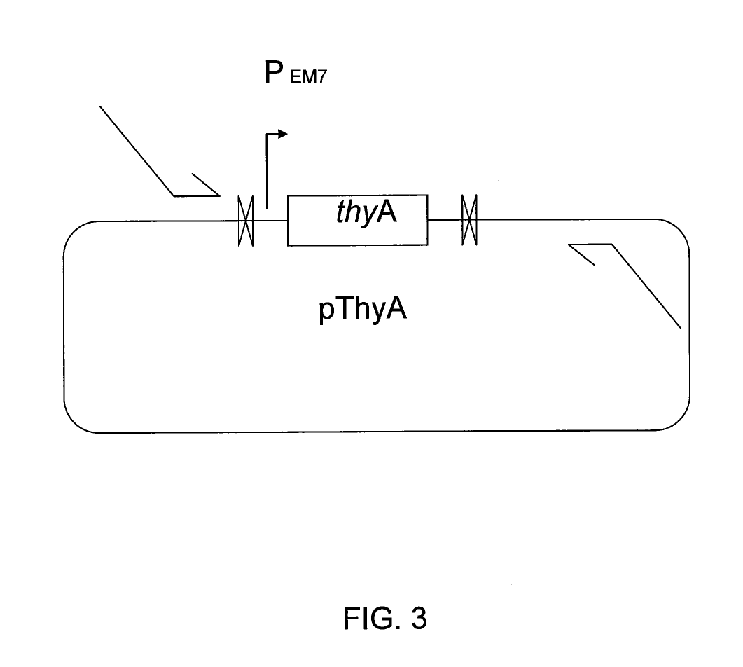

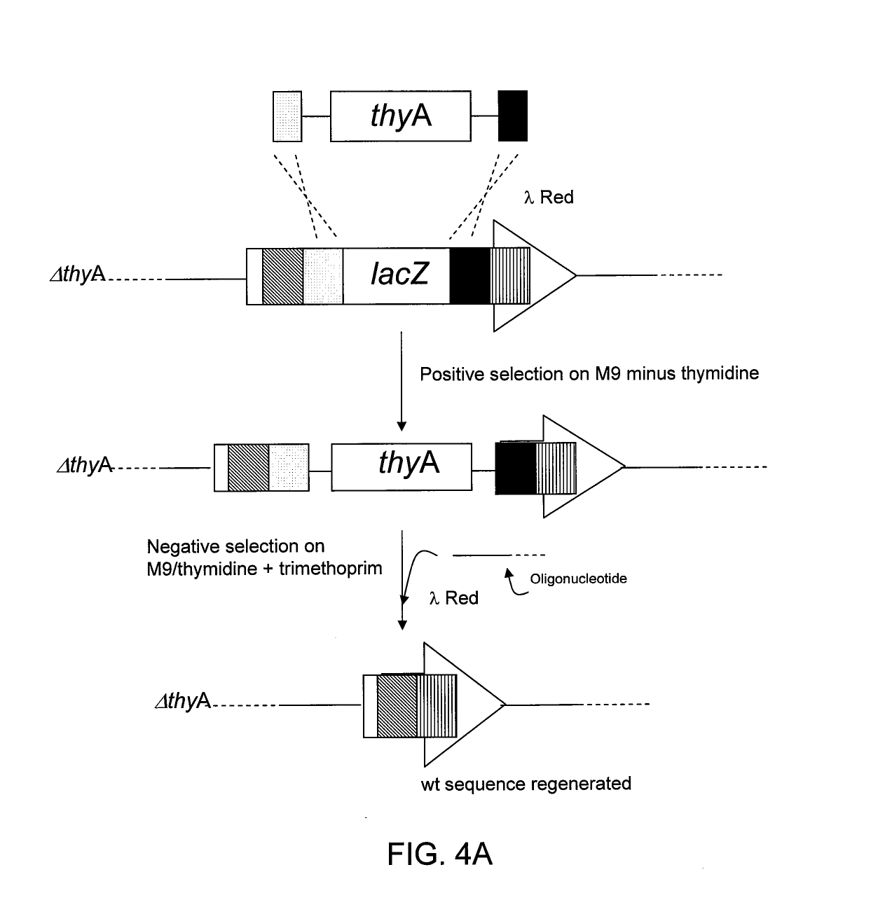

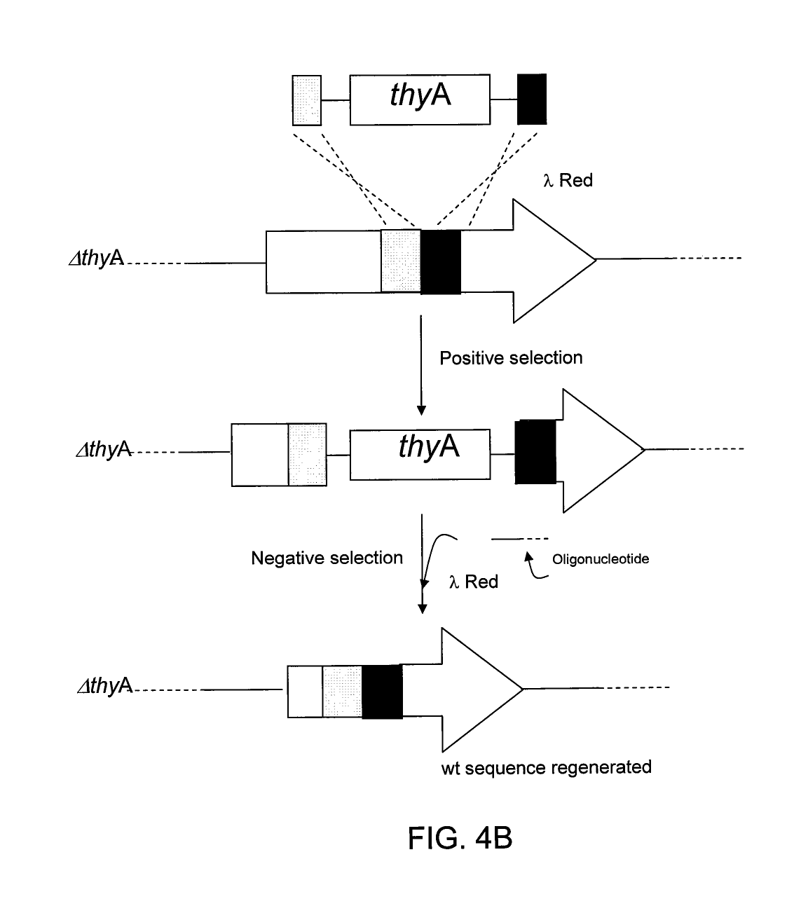

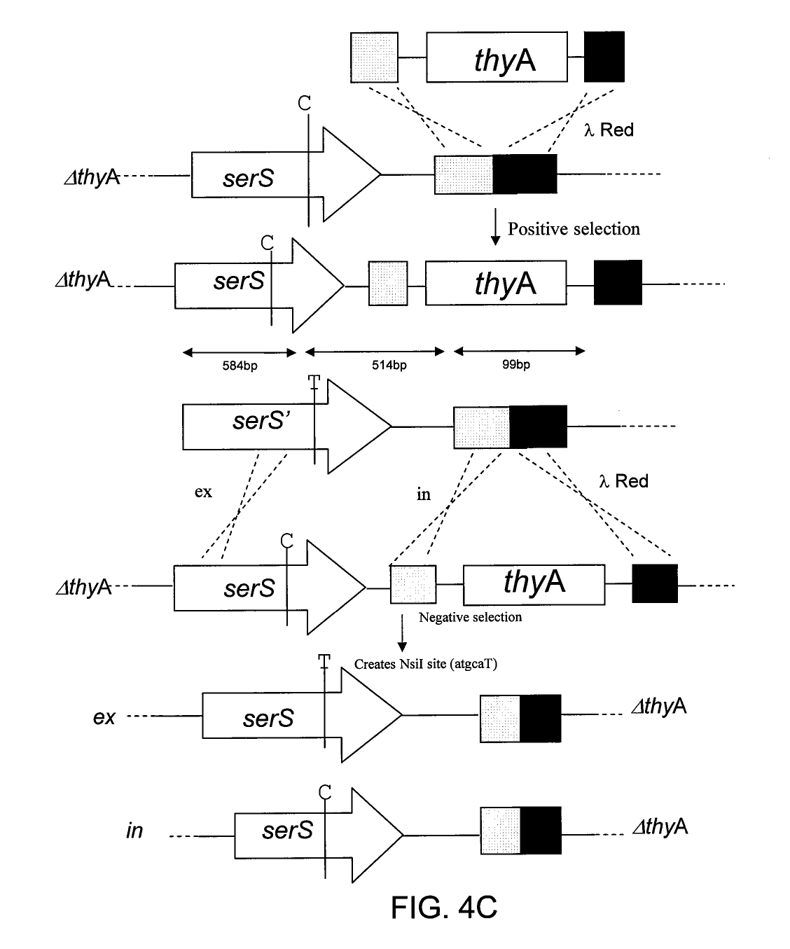

A PCR template, pThyA, containing thyA downstream of a strong constitutive promoter flanked by transcription terminators was constructed (set forth in FIG. 3). The entire unit is referred to herein as the “thyA cassette.” pThyA was used to generate 1.2 kb thyA cassettes targeted by 44 bp termini to the following loci: lacZ, thrA, trpC, tyrA, metC, and the serS downstream intergenic region. In each case, a two-step positive/negative thyA selection experiment was carried out in MG1655λ-ΔthyA-, in order to delete a nonessential gene (FIG. 4A), to simulate point mutation in a nonessential gene (FIG. 4B), and to simulate point mutation in an essential gene (serS) (FIG. 4C).

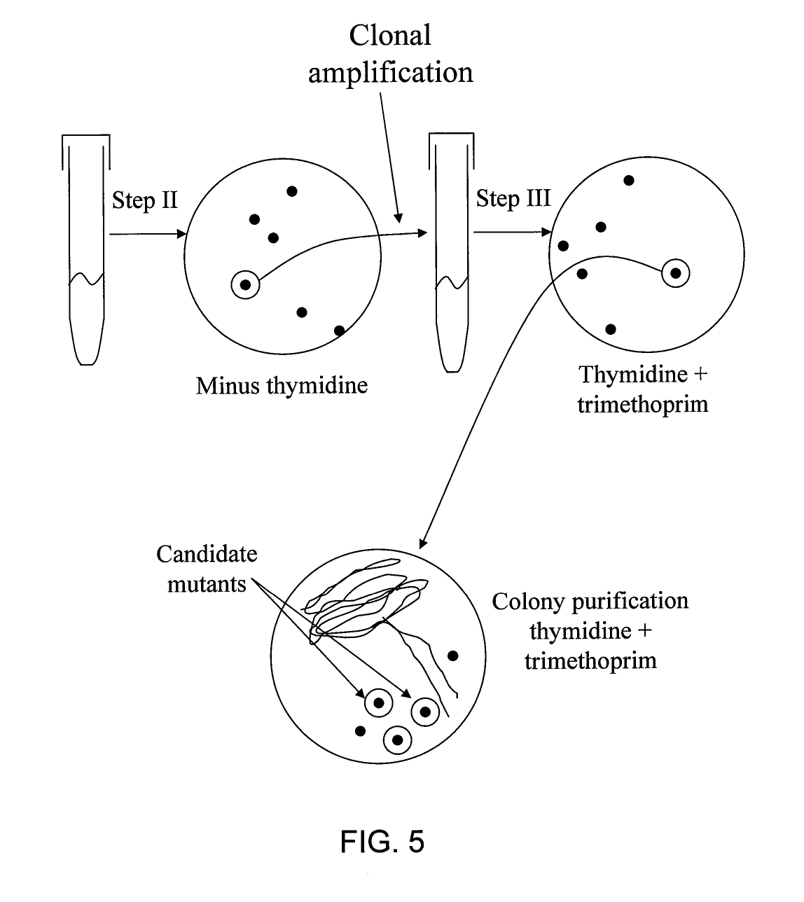

Using solid medium clonal selections throughout (see FIG. 5), a 100% (77/77) success rate in generating the desired mutations in the five nonessential genes was observed (Table 2). Although the thyA cassette was successfully evicted from the serS downstream region in 100% (14/14) of cases, a C→T substitution in the serS ORF was not obtained. Without intending to be bound by theory, this was likely due to internal recombination (see FIG. 4C).

Quantitative PCR (Q-PCR) (e.g., TAQMAN® and SYBR® Green protocols) may be used to further decrease homologous recombination at unintended sites. For example, Q-PCR may be used to amplify the introduced DNA up to the flanking sequences, using a bias equal to the ratio of correct/incorrect PCR products. Subsequent recombination with flanking primers should decrease frequency of unintended recombination.

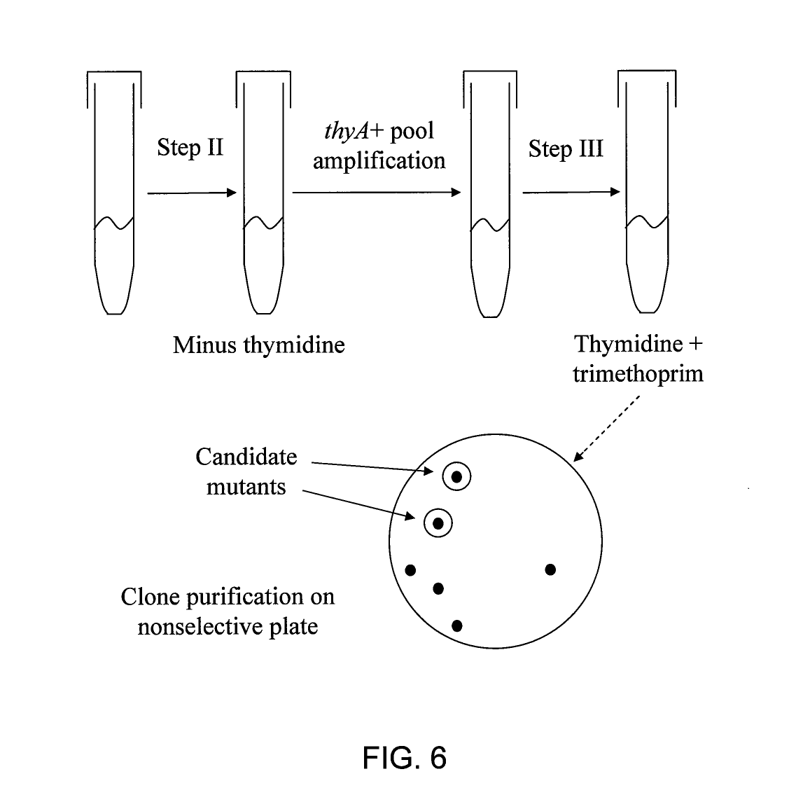

Conventional solid medium clonal selections used above were substituted by liquid medium non-clonal selections as shown in FIG. 6. The selections in liquid were less laborious than selections on plates. The construction of MG1655λ-ΔthyA-ΔlacZ (FIG. 4A) was repeated in liquid. 100% (84/84) of the candidate clones isolated on the final non-selective plates were confirmed to be ΔlacZ by diagnostic PCR. The results presented in Table 2 using gel-purified cassette PCRs are essentially free of any intact pThy.

TABLE 2

| Locus Targeted | Independent Colony-Purified Clones |
|---|---|
| lacZ | 20/20 |
| thrA | 12/12 |
| trpC | 15/15 |
| tyrA | 16/16 |
| metC | 14/14 |
| serS downstream | 14/14 evicted, but all wild-type |

The λ Red engineering methodology described above is particularly useful if one can be assured than the various assaults the target cell must undergo, such as thyA lesion, electroporation, induction of λ, and the like, do not increase the chromosomal spontaneous point mutation rate. A common way to query this rate is by measuring the rate of resistance to rifampicin (Rif^{R}), an antibiotic which binds to the β subunit of RNA polymerase encoded by rpoB. Essentially all known Rif^{R} mutations are base substitutions in rpoB.

The Rif^{R} frequency of MG1655 growing at 30° C. in Luria broth (LB) was compared to the Rif^{R} frequency of MG1655λ-ΔthyA growing in LB+thymidine. MG1655λ-ΔthyA had been subjected to a 10 minute heat shock at 42° C. to induce λ, electroporated in the presence of oligonucleotides, and plated on rich medium containing rifampicin (+thymidine for MG1655λ-ΔthyA). 18 independent cultures were tested for each strain. The Rif^{R} mutation rate was calculated according to the methods of Crane et al. (1996) Mutat. Res. 354:171, incorporated herein in its entirety for all purposes, using a modification of the Luria-Delbruck fluctuation analysis. λ Red induction in a ΔthyA background did not increase the Rif^{R} mutation rate. Table 3 depicts that no increase in spontaneous mutation rate was observed due to ΔthyA background and/or λ induction and electroporation (N=18 for MG1655 and MG1655λ-ΔthyA, N=1 for ES1578; ES1578 is a potent mutator strain and was included as a positive control; the mutation rate was calculated according to the protocol of Crane et al. (1996) Mutat Res. 22:171).

TABLE 3

| | | | Rif^{R} Mutation |
|---|---|---|---|
| Strain | λ Red Induction | Electroporation | Rate |
| MG1655 | No | No | 6.2 × 10⁻⁹ |
| MG1655λ-ΔthyA | Yes | Yes | 5.7 × 10⁻⁹ |
| ES1578 (mutD5) | No | No | 2.6 × 10⁻⁷ |

λ Red linear recombination is exceedingly efficient. Up to 1% of electroporated cells are recombinant when oligonucleotides are used (e.g. Step III of FIG. 1). One concern when considering its use in genome engineering is its propensity to recombine short tandem repeats that occur naturally throughout the E. coli chromosome. Without intending to be bound by theory, such cis recombination would be expected to generate chromosomal deletions at an increased frequency. To test this hypothesis, E. coli genome antisense arrays (Affymetrix, Inc. Santa Clara, Calif.) were used to compare the hybridization patterns of fluorescently labeled genomic DNA isolated from MG1655 and from MG1655λ-galK::cat, which is a clone in which a successful λ Red recombination had occurred such that the cat marker completely replaced galK. In effect, the aim was to perform a genomic Southern blot and scan for deleted regions in the λ Red-treated strain.

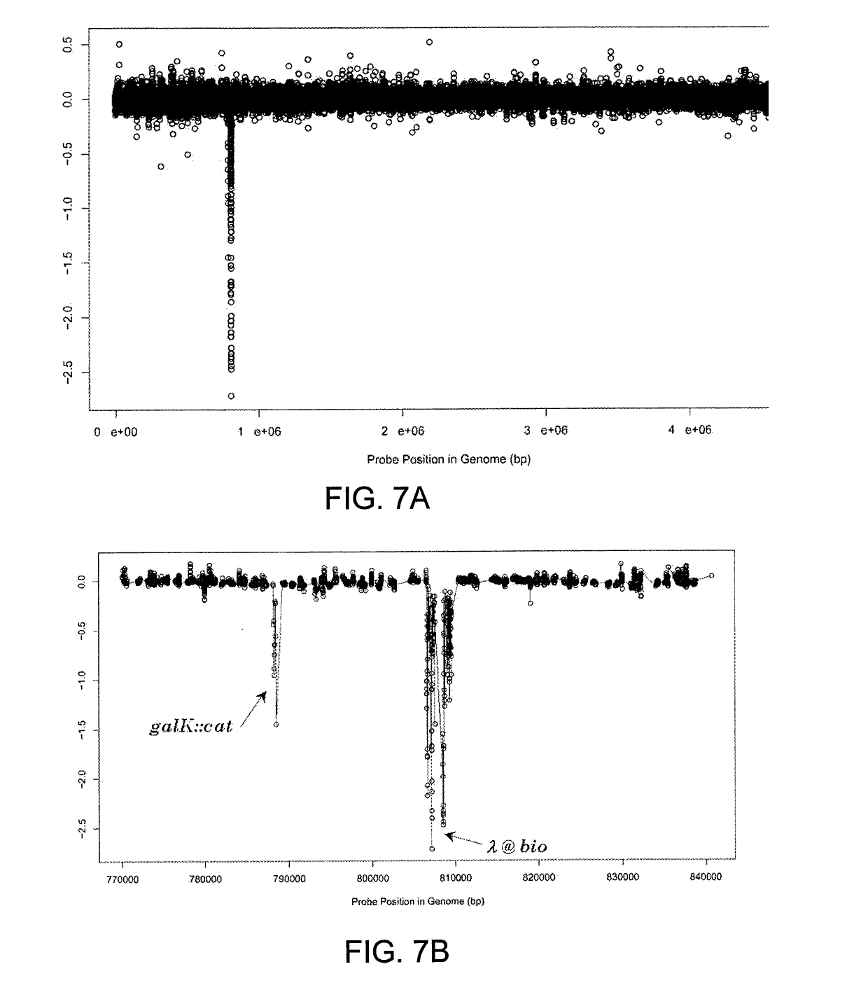

MG1655 and MG1655λ-galK::cat genomic DNAs were hybridized in triplicate. Affymetrix chip data processing was performed using the Affymetrix package (Bioconductor.org). Following background subtraction, quantile normalization, and averaging the “perfect-match” probe signals for all probes for each strain, the log [MG1655λ-galK::cat/MG1655] intensity ratio was calculated for each probe. By plotting R versus genomic probe position (FIGS. 7A-7B), regions deleted in MG1655λ-galK relative to MG1655 were identified as negative spikes projecting from a baseline of 0. λ Red recombination failed to induce any detectable chromosomal deletions outside of the desired galK::cat mutation. For MG1655λ, the λ genes replaced a large segment of the bio operon, indicating that the region was deleted.

### EXAMPLE III

### Serial Homologous Recombination

The combination of the lambda Red method of Yu et al., supra (incorporated herein by reference in its entirety for all purposes), and positive/negative selection with a single, doubly-selectable marker gene (e.g., thyA), as described herein, enables close to 100% correct homologous recombinants with little or no apparent associated damage to gross or fine chromosomal structure (See Example II, above). Integration of each desired novel subsequence into an acceptor DNA molecule in vivo requires two steps of electroporative recombination. Traditionally, for simple manipulations such as gene deletions, point mutations, and small insertions, the first thyA integration step occurs with a net efficiency of approximately 1×10⁻⁴, the second thyA counter-selective eviction step occurs with a net efficiency of approximately 1×10⁻³, and each of these steps uses approximately 1×10⁸ cells of E. coli.

Many fewer cells and smaller volume can be used for each electroporation. For example, 1×10⁶ cells in 10 microliters would still yield 100 cells before re-growth. Recovery after each step requires a one-hour incubation to permit expression/de-expression of the thyA, followed by at least two approximately 24-generation liquid amplifications of the positive/negative selectants (each amplification is approximately 18 hours in the defined medium required for the thyA selections, and nine hours for surface and GFP selections) (Schneckenburger et al. (2000) J. Biomed. Opt. 7:410; and Long (2004) Poster PG 25, University of Alabama at Birmingham; incorporated herein by reference in their entirety for all purposes).

It is desirable to minimize the time between cycles. One way to do this is by isolating in liquid the desired selectants without relying on a full batch growth cycle and/or two rounds of passaging. By selecting for mutated cells which have higher DNA uptake and integration rates and optimal corresponding growth and selection conditions, this can be achieved. For example, a variety of uptake/recombination/selection protocols known to those of skill in the art could be used to select for additional selectants. Selected mutants may then be manipulated using the same conditions used for the initial selection. Reducing the effective population 10,000-fold results in a requirement for more cell amplification. If the efficiency were 10% instead of 0.01% then much cell growth is not necessary. The devices described below would greatly aid such optimization.

### EXAMPLE IV

### Parallel Homologous Recombination

Methods of electroporation, optoporation, injection and chemically-enhanced transformation and/or transfection known in the art are restricted to containers such as cuvettes, microscope slides, microtitre plates and the like. It has been discovered that the use of a flow system such that the environment of the cell can be changed easily and a series of cells can be handled separated by space (and hence time) along a flowing stream. The flow can be regulated in one, two or three spatial dimensions by any combination of microfluidic forces known in the art, using methods such as electrophoresis, electroendoosmosis, capillary action, differential pressure, magnetic methods, optical methods, and the like. Many streams can be handled in parallel and intersected as needed for mixing. For instance, microfluidic manipulations of cells, such as those taught by Quake and Scherer ((2000) Science 290:1536, incorporated herein by reference in its entirety for all purposes) can be used for introducing DNA into cells.

The protocol described herein will enable one or more steps of high-throughput methods for introducing DNA into arrays of cells known in the art (such as those of Ziauddin and Sabatini (2001) Nature 411:107, incorporated herein by reference in its entirety for all purposes) to be automated. Further, micro hollow fibers (such as those taught in Gramer and Britton (2000) Hybridoma 19:407, incorporated herein by reference in its entirety for all purposes) can be used to exchange input and waste molecules including dissolved gases. With healthy cells, these systems can operate well without fouling with cell debris. Since electroporation, selection, or DNA preparation methods produce debris, however, cleaning cycles may be implemented or degradable and/or replaceable polymers may be utilized to line the microfluidic pathways.

### EXAMPLE V

### Multiplex Homologous Recombination

In addition to temporally-serial and spatially-parallel construction, multiplex constructions, i.e., multiple DNA introductions and selections occurring in the same population of cells in the same volume of medium, can be performed. Multiplexing can be enhanced by the use of multiple selectable/counter-selectable markers. In addition to thyA, selection can be achieved, for example, based on surface binding properties (U.S. Pat. No. 5,316,922, incorporated herein by reference in its entirety for all purposes) or by fluorescent cell sorting (Quake et al. (2000) Science 290:1536, incorporated herein by reference in its entirety for all purposes). Alternatively, other combinations of metabolites and inhibitors, e.g., proB/A in E. coli: positively selectable in a ΔproB/A genetic background and counter selectable in the presence of proline plus 4-nitropyridine 1-oxide may be used (Inuzuka et al. (1976) Antimicrob. Agents Chemother. 10:325, incorporated herein by reference in its entirety for all purposes).

DNA may be added exogenously, e.g., from elsewhere in the same microfluidic device such as, for example, from DNA chip release, by DNA error correction systems, via assembly functions and the like. DNA may also be added to cell suspension buffer by conventional means, such as micropipetting, or via dissolving a nucleic acid pellet, either before or after the buffer is run through the microfluidics device, and before the cells are added to the buffer. Alternatively, DNA from one cell can be moved into another cell via techniques such as mating, DNA isolation and re-transformation, the use of phage (e.g., P1) and the like. Thus, two or more DNA molecules from cells within the device can be recombined without the cells leaving the device.

The efficiency of this process can be improved by introducing double-stranded breaks in vivo (e.g., with SceI, see Posfai et al. (1999) Nucleic Acids Res. 27:4409, incorporated herein by reference in its entirety for all purposes) or in vitro. If multiple rounds of mating are needed, then two compatible conjugation systems can be used alternately. The choices of order of addition of DNA molecules can be predetermined or subject to automated phenotypic feedback based on the properties of the cell populations. Even though colony picking is not required, the option to establish clonal derivatives within the device by dilution or single-cell-sorting is considered another positive feature of the system described herein.

### EXAMPLE VI

### Global Replacement of Codons

This example provides for a novel tRNA synthetase which uses novel amino acids to be supplied in the growth medium or synthesized internally (e.g., either by engineering a novel tRNA synthetase, or by introducing an exogenous tRNA synthetase into a cell). This requires tRNA synthetase-tRNA pairs which do not cross-react with the core set. For example, a glutamate pair from Pyrococcus horikoshii, and a tyrosine pair from Methanococcus jannaschii may be used (described in Chin et al. (2003) Science 2003 301:964; and Santoro et al. (2003) Nucleic Acids Res. 31:6700; incorporated herein by reference in their entirety for all purposes). These are typically usable in other organisms only for the stop codon, UGA, or in vitro. Replacing one or more of the 61 coding E. coli codon(s) genome-wide could mean 4000 gene replacements (of 1 kb each). While this may be performed using only one gene chip, many transformations, e.g., 2 per day for 2000 days, would be necessary. The present invention provides multiplex, hierarchical and serial recombination techniques that could be used to greatly decrease this timeframe. For example, the larger the construct, the more mutations can be performed in parallel. Increasing oligo size from 1 kb to 20 kb would reduce the time necessary for replacing the codons from 2000 days to 100 days. Using bacterial artificial chromosomes (BACs) that are 300 kb in length, this timeframe could be further reduced down to 20 days. Without intending to be bound by theory, the final and/or intermediate genomes obtained by the process described herein should be viable. The genome sequence may be confirmed using methods that include, but are not limited to, whole genome sequencing and chip hybridization (see, for example, FIG. 7).

Without intending to be bound by theory, one way to predictably engineer existing tRNA synthetases to alter their substrate specificity and to allow an existing tRNA to carry a different amino acid is as follows. The tRNA-synthetases (S) and tRNAs (T) will be mutated and expressed in cis on the same plasmid. The mutated S and T genes will be transcribed and translated in an emulsion containing all factors necessary for translation except for the native S and T. If the mutated S recognizes the mutated T, the nascent S-chain will form. Ribosome display methods will then be used to select for mRNA encoding such an S and T pair. Counter-selection against S that still recognizes an unmodified tRNA(U), for example, may be performed by including a non-acylatable competitor U in the emulsion or by second step selection against S to U binding in vitro.

### EXAMPLE VII

### Codon Swapping

If two or more codons are replaced as in Example VI, an alternative to using them for novel amino acids is to use them with standard amino acids but using novel combinations of amino acids and codons. This could provide resistance to viruses and provide a barrier to genetic exchange with wild-type species. Such resistance is desirable, for instance, in the development of genetically modified organisms and exotic microbial strains. For example, Leu, Ser and Ala synthetases do not recognize the anticodon, so switches among their sixteen codons could be achieved.

Replacement of codons may be performed as follows. Without intending to be bound by theory, the codons UUR (wherein R=A or G) are normally recognized by the Leu tRNA synthetases, while the codons AGY (wherein Y=C or T) are normally recognized by Ser tRNA synthetases. All of the UUR codons in the genome will be converted to CUX codons (wherein X=G, A, T or C), which normally encode Leu via a different set of tRNAs. All of the AGY codons in the genome will be changed to UCX codons, which encode Ser via a different set of tRNAs. Next, the Leu-tRNAs' anticodons specific to UUR will be changed to recognize AGY, and the Ser-tRNAs' anticodons specific for AGY will be changed to UUR (an anticodon swap between tRNAs which are at the time unused). Next, all of the CUX codons in the genome will be changed to AGY. Finally, all of the UCX codons in the genome will be changed to UUR.

### EXAMPLE VIII

### Restriction Site Removal

Taking advantage of neutral (as regards amino acid coding) substitutions, out of the four to eight specificity-determining bases of each site, at least one can be changed. Once all of the sites are removed, the genome is resistant to restriction endonucleases even in the absence of the cognate DNA-methylase. Accordingly, restriction reactions on specialized DNA components such as plasmids can occur without affecting the integrity of the main chromosome. Various type IIS endonuclease (e.g., AarI, SapI, EcoPI, BsaI, BsmBI, EarI, MmeI, FokI, HgaI, BsmFI, MboII, HphI and the like) strategies, such as VDJ-like DNA memory (U.S. Ser. No. 10/427,745, incorporated herein by reference in its entirety for all purposes) or in vivo DNA computing (Benenson et al. (2004) Nature 429:423, incorporated herein by reference in its entirety for all purposes) may be used.

### EXAMPLE IX

### Human Stem Cells

Analogous to the surface selection for bacteria described herein, similar selections can be executed on mammalian cells (e.g., human cells or mouse cells). For example, to reduce or eliminate the T-cell-mediated response that causes graft rejection, “generic” human embryonic stem cells with desirable natural or engineered traits may be used to make a variety of derivatives which are homozygous (or heterozygous) for various major and minor histocompatibility antigens. Examples of major antigens include, but are not limited to, the nine major histocompatibility complexes (MHCs): HLA-A, HLA-B, HLA-C, HLA-DPA1, HLA-DPB1, HLA-DQA1, HLA-DQB1, HLA-DRA, and HLA-DRB1. Examples of minor antigens include, but are not limited to HA-1, HB-1, HA-2 (myosin MYO1G), UGT2B17 (i.e., deletion of one gene of a multi-gene family), MiHA (murine mitochondrial protein COI) and the like. The advantage of homozygous cells is that one cell line can be useful for a larger number of people, while heterozygous cells will be close to unique. Another alternative is serial/parallel introduction of the “generic” modifications into stem cells from each individual patient. These stem cells could be obtained from various adult stem cell populations or by cell-fusion or nuclear transplantation from any cell into appropriate generic human embryonic stem cells or embryonic germ cells (Hubner et al. (2003) Science 300:1251, incorporated herein by reference in its entirety for all purposes). Similarly, nuclear transplantation into oocytes and/or transfer into germ cells derived from embryonic stem cells using methods known in the art would be useful for modifying mammalian cells.

### EXAMPLE X

### Materials and Methods

MG1655ΔthyA λRed (EcNR2) is a bacterial strain that contains a defective λ Red prophage at the bio locus, and accordingly supplies all recombination proteins under control of a temperature-inducible promoter (Yu et al., supra, incorporated herein by reference in its entirety for all purposes). This strain was constructed by (1) recombineering the bla gene into the right terminus of the λ Red prophage in DY330; (2) P1 transduction of the modified prophage unit into MG1655 by virtue of its conferral of carbenicillin resistance; and (3) deletion of the native thyA gene in the MG1655 transductant by recombining with a ‘knock-out’ thyA-targeted oligonucleotide (5′-CTGGTGACAACTAAACGTTGCCACCTGCGTTCCATTA ATTACGAAACATCCTGCCAGAGCCGACGCCAGT (SEQ ID NO:1)) and selection on M9 minimal medium containing 100 μg/mL thymidine plus 4 μg/mL trimethoprim. EcNR2 is auxotrophic for both thymine/thymidine and biotin. It and any derivatives of it that did not contain a functional copy of thyA were routinely maintained in 100 μg/mL thymidine. Biotin was always included at a concentration of 0.25 μg/mL. All engineering operations comprised the following steps: (1) growth of EcNR2 parent/derivative strain to exponential phase at 30° C.; (2) induction of λ Red prophage by shifting to 30° C. for 12 minutes; (3) ice-chilling; (4) washing cells in at least two volumes of double-distilled water; (5) electroporation of cells (at ˜10¹⁰ cells/mL) with the linear double-stranded or single-stranded DNA of interest at 12.5 kV/cm, 200Ω, 25 μF; (6) recovery in rich medium with thymidine for one hour at 30° C.; and (7) selective amplification of recombinants at 30° C. by growth in or on the appropriate liquid or solid medium at 30° C. For positive selection, the medium used was rich defined medium (e.g., Neidhardt Supplemented MOPS Defined Medium lacking any source of thymine or thymidine (Neidhardt et al. (1974) J. Bacteriol. 119:736, available commercially from TekNova Inc., Half Moon Bay, Calif.). For negative selections, the medium used was the same rich defined medium containing 100 μg/mL thymidine plus 4-20 μg/mL trimethoprim. For most applications, one milliliter of log phase cells was used per electroporation. Using 0.1-1 μg linear DNA, one can typically expect 10³-10⁵ desired (counter)selected recombinants. For all selections, a range of dilutions of the recovery mix was typically used to inoculate liquid or plate cultures.

The sequence of the thyA cassette region of pThyA, i.e., the thyA gene from L. lactis under control of a constitutive promoter, flanked by two different bidirectional transcriptional terminators:

(SEQ ID NO:2)

GCGGCCGCTCTAAAAAATGCCCTCTTGGGTTATCAAGAGGGTCATTATAT
TTCGCGGAATTCATGCTATCGACGTCTGATATCAGAGCTCTGTTGACAAT
TAATCATCGGCATAGTATATCGGCATAGTATAATACGACAAGGTGAGGAA
CTAAACCATGACTTACGCAGACAAGATTTTTAAACAAAATATCCAAAATA
TCCTTGATAACGGTGTTTTTTCAGAAAATGCACGTCCCAAGTATAAGGAT
GGTCAAACCGCAAATAGTAAGTATGTAACAGGCTCTTTCGTTACTTATGA
TTTGCAAAAAGGTGAGTTTCCAATTACCACTTTGCGTCCAATTCCAATTA
AATCGGCAATTAAAGAATTGATGTGGATTTATCAAGATCAAACAAGTGAA
CTTGCTATTCTTGAAGAAAAATATGGAGTCAAATACTGGGGCGAATGGGG
AATTGGCGACGGTACGATTGGACAACGCTATGGGGCAACAGTCAAAAAAT
ATAATATTATTGGAAAATTATTGGATGGTTTAGCAAAAAATCCTTGGAAT
CGCCGTAATATCATTAATCTTTGGCAATATGAAGATTTTGAGGAAACTGA
AGGACTTTTGCCTTGTGCTTTCCAAACAATGTTTGATGTTCGCCGTGAAC
AAGATGGTCAGATTTACTTGGATGCGACTCTTATTCAACGTTCAAATGAT
ATGCTTGTTGCGCATCATATCAATGCCATGCAATATGTTGCTTTACAAAT
GATGATTGCAAAACATTTTTCTTGGAAAGTTGGAAAATTTTTCTATTTCG
TAAATAATTTGCATATTTATGATAATCAGTTTGAACAGGCAAATGAGTTA
GTTAAGCGAACAGCTTCTGACAAGGAGCCCCGTTTGGTGCTTAATGTTCC
TGACGGAACAAACTTTTTCGATATTAAACCAGAAGATTTTGAGCTTGTGG
ACTATGAACCGGTAAAACCTCAATTAAAATTTGATTTGGCAATTTAGGCG
CGCCTGTAATCACACTGGCTCACCTTCGGGTGGGCCTTTCTGCTTTAAAA
AAAACGGGCCGGCGCGAACGCCGGCCCGCGGCCGC.

This unit was constructed by PCR using L. lactis genomic DNA as template and inserted into a PBLUESCRIPT® (Stratagene, La Jolla, Calif.) vector backbone for sequence verification.

Double-stranded targeting thyA cassettes for positive selection steps were generated by PCR using salt-free 69-mers, each comprising 23 nucleotides of priming homology to pThyA and 46 nucleotides of target-specific sequence. To obtain a lacZ deletion, for example, the following PCR primers would be utilized with a pThyA template:

forward:

(SEQ ID NO:3)

5′-ACCATGATTACGGATTCACTGGCCGTCGTTTTACAACGTCGTGACTg
ctgatccactagttctagagcg
reverse:

(SEQ ID NO:4)

5′-TTTTTGACACCAGACCAACTGGTAATGGTAGCGACCGGCGCTCAGCc
taaagggaacaaaagctgggtg

Lowercase bases indicate priming sequences, uppercase targeting ones. PCR products (1.2 kb) were purified using QIAQUICK® PCR columns (QIAGEN Inc., Valencia Calif.) and eluted in 1 mM Tris (pH 8.0). Single-stranded targeting DNAs used for negative selection steps were salt-free synthetic oligonucleotides, usually 70 nucleotides in length, having approximately 35 nucleotides of homology on each side (see FIG. 4A).

After all serial engineering steps were carried out for a given strain of interest, the ΔthyA locus was repaired to its wild-type thyA status by recombining with a cloned double-stranded restriction fragment comprising the wild-type thyA gene flanked by 100 base pairs of adjacent homology on each side, following positive selection as described above. The final step was to remove the integrated λ Red prophage. This was done by recombining with a double-stranded fragment encompassing the bio genes deleted in the original P1 transduction via approximately 300 base pairs of terminal homology. Successful recombinogenic removal of the prophage was positively selectable because these recombinants were able to grow in the absence of biotin supplementation, i.e., the selection medium was rich defined medium lacking biotin. The sequences of these ΔthyA and λ removal fragments are shown:

ΔthyA removal

(SEQ ID NO:5)

GCCGGCGGTCATCAGATGCGTTTTAACCTGCAAGATGGATTCCCGCTGGT
GACAACTAAACGTTGCCACCTGCGTTCCATCATCCATGAACTGCTGTGGT
TTCTGCAGGGCGACACTAACATTGCTTATCTACACGAAAACAATGTCACC
ATCTGGGACGAATGGGCCGATGAAAACGGCGACCTCGGGCCAGTGTATGG
TAAACAGTGGCGCGCCTGGCCAACGCCAGATGGTCGTCATATTGACCAGA
TCACTACGGTACTGAACCAGCTGAAAAACGACCCGGATTCGCGCCGCATT
ATTGTTTCAGCGTGGAACGTAGGCGAACTGGATAAAATGGCGCTGGCACC
GTGCCATGCATTCTTCCAGTTCTATGTGGCAGACGGCAAACTCTCTTGCC
AGCTTTATCAGCGCTCCTGTGACGTCTTCCTCGGCCTGCCGTTCAACATT
GCCAGCTACGCGTTATTGGTGCATATGATGGCGCAGCAGTGCGATCTGGA
AGTGGGTGATTTTGTCTGGACCGGTGGCGACACGCATCTGTACAGCAACC
ATATGGATCAAACTCATCTGCAATTAAGCCGCGAACCGCGTCCGCTGCCG
AAGTTGATTATCAAACGTAAACCCGAATCCATCTTCGACTACCGTTTCGA
AGACTTTGAGATTGAAGGCTACGATCCGCATCCGGGCATTAAAGCGCCGG
TGGCTATCTAATTACGAAACATCCTGCCAGAGCCGACGCCAGTGTGCGTC
GGTTTTTTTACCCTCCGTTAAATTCTTCGAGACGCCTTCCGCCGGC
λ removal

(SEQ ID NO:6)

GCCGGCAGGGTAATCGGGGAAGGATTCCACGCTGCAGCACCTGGTGTCAG
GGATGCAAAATAGTGTTGAGCATCGAAATTCTGCGCTTCTTTTGCCGACA
GAATCGGGCGAGAAGAGGTACCAGGCGCGGTTTGATCAGAAGGACGTTGA
TCGGGCGGGGTTGAGCTACAGGCGGTCAGCGTCACGCCAAAAGCCAATGC
CAGCGCCAGACGGGAAACTGAAAATGTGTTCACAGGTTGCTCCGGGCTAT
GAAATAGAAAAATGAATCCGTTGAAGCCTGCTTTTTTATACTAACTTGAG
CGAAACGGGAAGGTAAAAAGACAAAAAGTTGTTTTTAATACCTTTAAGTG
ATACCAGATGGCATTGCGCCATCTGGCAGAGTGATTAACTAAACATCGCA
GTAATCGAGGCGCTTGCCAGAGAGTGGAAATGAACGTTAAACCCGACCAT
CGCGCCGCTGGCACCTTCATCGACATCAATACGTTCTATATCCAGCGCGT
GAACGGTAAAAATGTAGCGATGAGTTTCGCCTTTCGGCGGTGCTGCGCCA
TCGTACCCGGTTTTACCAAAGTCGGTACGCGTCTGCAAAACGCCGTCTGG
CATTGCTACCAGACCAGAGCCAAACCCTTGCGGTAATACGCGGGTATCAG
CGGGTAAGTTAACAACTACCCAGTGCCACCAGCCGGAGCCGGTTGGCGCA
TCCGGGTCGTAGCAGGTGACAACAAAACTTTTCGTTCCCGCAGGAACATC
ATCCCACGCCAGATGCGGTGAAATATTATCGCCATCGTAACCCATGCCGT
TAAAGACATGACGATGCGGCAATTTATCGCCATCGCGCAGATCGTTACTG
ATGAGTTTCATGAACCCTCCTTTCTTGTTTGCAGAAAGTGTAGCCAGAAA
CCCTCACGCGGACTTCTCGTTATTGGCAAAAAAATGTTTCATCCTGTACC
GCGCGGTTAACCGCTGCGGTCAGACGCTGCAACTGTTGCGGGAGAATAAT
ATAGGGCGGCATCAGGTAAATCAGTTTGCCAAAAGGCCGGATCCAGACAC
CCTGTTCGACAAAGAATTTTTGCAGCGCCGCCATATTCACCGGATGAGTG
GTTTCGACCACGCCAATGGCCCCCAGTACGCGCACATCGGCAACCATTTC
GGCATCACGGGCGGGGGCAAGTTGCTCGCGCAGCTGTACTTCAATATCCG
CCACCTGTTGCTGCCAGTCGCCAGATTCGAGAATCGCCAGGCTGGCGTTT
GCTGCCGCGCAGGCCAGCGGATTGCCCATAAAAGTTGGCCCATGCATAAA
GCAACCGGCTTCACCGTTACTGATGGTTTCTGCAACCTCGCGCGTGGTGA
GTGTGGCGGAAAGGGTCATTGTGCCGCCGGTTAAGGCTTTACCGAGGCAC
AAAATGTCCGGCGCGATTTCTGCATGTTCACAGGCAAACAGTTTCCCGGT
ACGACCAAATCCAGTGGCGATCTCGTCGGCAATCAGCAAGATACCTTCGC
GATCGCATATTTTGCGGATTCGTTTTAACCATTCCGGATGGTACATGCGC
ATCCCGCCTGCGCCCTGGACAATCGGCTCAATGATCACCGCCGCGATTTC
ATGACGATGCGCCGCCATCAGGCGGGCAAAGCCCACCATATCGCGCTCAT
CCCATTCGCCATCCATGCGGCTTTGCGGGGCGGGAGCAAACAGGTTTTCT
GGCAGGTAGCCTTTCCACAGACTGTGCATTGAGTTATCCGGATCGCACAC
CGACATCGCGCCAAAGGTATCGCCATGATAACCATTGCGGAAGGTCAGAA
AACGCTGGCGCGCTTCGCCTTTGGCTTGCCAGTACTGCAACGCCATTTTC
ATCGCCACTTCCACCGCTACGGAACCGGAGTCCGCGAGAAAAACGCACTC
CAGCGGTTGCGGCGTCATCGCCACCAGTTTGCGGCACAGCTCAATGGCTG
GCGCATGGGTGATACCGCCAAACATCACATGCGACATGGCATCAATTTGC
GACTTCATCGCCGCATTAAGCTGCGGGTGATTGTAGCCGTGGATCGCCGC
CCACCAGGACGACATACCGTCAACCAGGCGTCTGCCGTCAGACAAAATCA
GCTCGCAACCTTCGGCGCTCACCACCGGATAAACCGGCAGAGGGGAGGTC
ATGGATGTGTATGGGTGCCAGATATGGCGTTGGTCAAAGGCAAGATCGTC
CGTTGTCATAATCGACTTGTAAACCAAATTGAAAAGATTTAGGTTTACAA
GTCTACACCGAATTAACAACAAAAAACACGTTTTGGAGAAGCCCCATGGC
TCACCGCCCACGCTGGACATTGTCGCAAGTCACAGAATTATTTGAAAAAC
CGTTGCTGGATCTGCTGTTTGAAGCGCAGCAGGTGCATCGCCAGCATTTC
GATCCTCGTCAGGTGCAGGTCAGCACGTTGCTGTCGATTAAGACCGGAGC
TTGTCCGGAAGATTGCAAATACTGCCCGCAAAGCTCGCGCTACAAAACCG
GGCTGGAAGCCGAGCGGTTGATGGAAGTTGAACAGGTGCTGGAGTCGGCG
CGCAAAGCGAAAGCGGCAGGATCGACGCGCTTCTGTATGGGCGCGGCGTG
GAAGAATCCCCACGAACGCGATATGCCGTACCTGGAACAAATGGTGCAGG
GGGTAAAAGCGATGGGGCTGGAGGCGTGTATGACGCTGGGCACGTTGAGT
GAATCTCAGGCGCAGCGCCTCGCGAACGCCGGGCTGGATTACTACAACCA
CAACCTGGACACCTCGCCGGAGTTTTACGGCAATATCATCACCACACGCA
CTTATCAGGAACGCCTCGATACGCTGGAAAAAGTGCGCGATGCCGGGATC
AAAGTCTGTTCTGGCGGCATTGTGGGCTTAGGCGAAACGGTAAAAGATCG
CGCCGGATTATTGCTGCAACTGGCAAACCTGCCGACGCCGCCGGAAAGCG
TGCCAATCAACATGCTGGTGAAGGTGAAAGGCACGCCGCTTGCCGATAAC
GATGATGTCGATGCCTTTGATTTTATTCGCACCATTGCGGTCGCGCGGAT
CATGATGCCAACCTCTTACGTGCGCCTTTCTGCCGGACGCGAGCAGATGA
ACGAACAGACTCAGGCGATGTGCTTTATGGCAGGCGCAAACTCGATTTTC
TACGGTTGCAAACTGCTGACCACGCCGAATCCGGAAGAAGATAAAGACCT
GCAACTGTTCCGCAAACTGGGGCTAAATCCGCAGCAAACTGCCGTGCTGG
CAGGGGATAACGAACAACAGCAACGTCTTGAACAGGCGCTGATGACCCCG
GACACCGACGAATATTACAACGCGGCAGCATTATGAGCTGGCAGGAGAAA
ATCAACGCGGCGCTCGATGCGCGGCGTGCTGCCGATGCCCTGCGTCGCCG
TTATCCGGTGGCGCAAGGAGCCGGACGCTGGCTGGTGGCGGATGATCGCC
AGTATCTGAACTTTTCCAGTAACGATTATTTAGGTTTAAGCCATCATCCG
CAAATTATCCGTGCCTGGCAGCAGGGGGCGGAGCAATTTGGCATCGGTAG
CGGCGGCTCCGGTCACGTCAGCGGTTATAGCGTGGTGCATCAGGCACTGG
AAGAAGAGCTGGCCGGC

SEQ ID Nos:5 and 6 each represent a gel-purified NgoMIV restriction fragment from the appropriate plasmid.

### EXAMPLE XI

### Comparison of thyA Positive/Negative Selection (T+/T−) in E. coli and kanMX Positive/URA3 Negative Selection (K+/U−) in S. cerevisiae

Efficiency

thyA (T+) is more efficient than kanMX (K+). Depending on how the locus was targeted, 50 ng of a PCR-generated thyA cassette having approximately 50 base pair terminal homologies yielded approximately 1×10³ to 1×10⁵ thyA+ recombinants per 1×10⁸ viable cells in T+. In contrast, approximately 3 μg (i.e., 3,000 ng) of a similarly generated K+ cassette yielded only 1×10¹ to 1×10² G418-resistant recombinants per 1×10⁸ viable cells. Thus, the normalized efficiency in number of recombinants per 1×10⁸ viable cells per mg of cassette with short terminal homologies is 2×10⁴ to 2×10⁶: 3×10⁰ to 3×10¹ (T+: K+). These data correspond to an increased efficiency of 700-fold to 700,000-fold.

Furthermore, the T+/− system described herein relies upon recombination that occurs rapidly (i.e., less than 15 minutes), whereas the S. cerevisiae system is naturally recombinogenic with respect to linear DNA molecules

Accuracy

T+ is more accurate than K+. Approximately 1×10³ T+-type recombinations have been screened across a spectrum of loci without observing an incorrectly integrated thyA cassette. Accordingly, T+ cells need not be screened for correct integration events. In contrast, K+ is thought to have an efficiency on the order of approximately 75% accuracy. Indeed, the delitto perfetto protocol of Storici et al. (supra) requires an essential screening step to obtain positively-selected integrants.

Robustness

T− is more robust than U−. In delitto perfetto (supra), replica plating is necessary to obtain a subset of 5FOA-resistant cells that are also G418-sensitive. Using the delitto perfetto system, a primary reason for the need to screen for U− is that there is no means of selecting for URA3 functionality following K+. A significant percentage of G418-resistant K+ recombinants have a PCR-mutated and/or partially functional URA3 gene.

For T−, if one desires to select transformants by plating, one need only plate on thymidine/trimethoprim plates and select faster growing (i.e., larger) colonies. In contrast to the U− system above, because thyA serves as both a positive and a negative selection marker, any thyA+ recombinant will also have a functional, i.e., counter-selectable, thyA.

### Equivalents

Other embodiments will be evident to those of skill in the art. It should be understood that the foregoing description is provided for clarity only and is merely exemplary. The spirit and scope of the present invention are not limited to the above examples, but are encompassed by the claims. All publications, patents and patent applications cited above are incorporated by reference herein in their entirety for all purposes to the same extent as if each individual publication or patent application were specifically indicated to be so incorporated by reference.

### ADDITIONAL REFERENCES

These references are incorporated herein by reference in their entirety for all purposes.
Belteki et al. (2003) Nat. Biotechnol. 21:321.
Benhar (2001) Biotechnol. Adv. 19:1.
Church et al., U.S. Ser. No. 60/548,637 and U.S. Ser. No. 60/600,957
Gramer et al. (2000) Hybridoma 19:407
Fuhrmann et al. (2003) Science 300:1251
Kolisnychenko et al. (2002) Genome Res. 12:640
Link et al. (1997) J. Bacteriol. 179:6228
Popov et al. (1996) Gene 177:195
Posfai et al. (1999) Nucleic Acids Res. 27:4409
Storici et al. (2001) Nat. Biotechnol. 19:773
Valenzuela et al. (2003) Nat. Biotechnol. 21:652
Yang et al. (2003) Nat. Biotechnol. 21:447
Zwaka et al. (2003) Nat. Biotechnol. 21:319
Edwards et al. (2000) Proc. Natl. Acad. Sci. U.S.A. 97:5528
Inuzuka et al. (1976) Antimicrob. Agents Chemother. 10:333
Boeke et al. (1984) Mol. Gen. Genet. 197:345
Segre et al. (2002) Proc. Natl. Acad. Sci. U.S.A. 99:15112
Dale et al. (1972) J. Bacteriol. 110:905
Langle-Rouault et al. (1995) Nucleic Acids Res. 23:3079

What is claimed is:

1. A method of in vivo serial alteration of a nucleic acid sequence of interest in a target cell comprising the steps of:
a. contacting a sample of one or more target cells with a first exogenous nucleic acid sequence having a selection marker, and allowing the first exogenous nucleic acid sequence to exchange with a nucleic acid sequence in target cells in the sample to produce first recombinant target cells;
b. contacting the sample of one or more target cells resulting from step (a) with a first selection substrate, wherein the first recombinant target cells are selected if they express the selection marker to produce a sample of first selected recombinant target cells;
c. contacting the sample of one or more target cells resulting from step (b) with a second exogenous nucleic acid sequence, and allowing the second exogenous nucleic acid sequence to exchange with a nucleic acid sequence in the one or more first selected recombinant target cells to produce a sample of one or more further recombinant target cells; and
d. contacting the sample of one or more target cells resulting from step (c) with a second selection substrate, wherein the one or more further recombinant target cells are selected if they lack expression of the selection marker, thereby providing a sample of further recombinant target cells.

2. The method of claim 1, wherein the selection marker comprises a single selection component.

3. The method of claim 2, wherein the selection component is thyA.

4. The method of claim 1, wherein the selection marker comprises a positive selection component and a negative selection component.

5. The method of claim 4, wherein the positive selection component is a member selected from the group consisting of a visually detectable marker, a marker that allows growth in media lacking a nutrient, and a marker that confers resistance to a compound.

6. The method of claim 4, wherein the negative selection component is a visually detectable marker or a marker that is lethal to the cell in the presence of substrate.

7. The method of claim 1, wherein the first selection substrate comprises a minimal medium, a medium that lacks one or more nutrients essential to the one or more target cells or an antibiotic.

8. The method of claim 1, wherein the one or more target cells are altered by addition to their genome of one or more nucleotides.

9. The method of claim 1, wherein the one or more target cells are altered by removal of one or more nucleotides from their genome.

10. The method of claim 1, wherein the one or more target cells are altered by exchange of one or more nucleotides in their genome.

11. The method of claim 1, wherein the one or more target cells are altered by one or more of addition to their genome of one or more nucleotides, removal of one or more nucleotides from their genome and exchange of one or more nucleotides in their genome.

12. The method of claim 1, wherein alteration of the genome of the one or more target cells is made to one or more of an endogenous gene, an endogenous intergenic nucleic acid sequence, and a non-geneic nucleic acid sequence.

13. The method of claim 1, wherein an amino acid specificity of a tRNA synthetase is altered.

14. The method of claim 11, wherein the amino acid specificity of a tRNA synthetase is altered to utilize a different amino acid.

15. The method of claim 11, wherein a novel tRNA synthetase is expressed in the cell.

16. The method of claim 11, wherein an exogenous tRNA is expressed in the cell.

17. The method of claim 1, wherein a restriction endonuclease cleavage site is altered.

18. The method of claim 1, wherein the target cell is selected from the group consisting of a bacterial cell, a yeast cell, a plant cell, an insect cell, an amphibian cell, a nematode cell and a mammalian cell.

19. A method of in vivo serial alteration of a nucleic acid sequence of interest in a target cell comprising the steps of:
a. contacting a sample of one or more target cells with a first exogenous nucleic acid sequence having a first selection marker and a second selection marker, and allowing the first exogenous nucleic acid sequence to exchange with a nucleic acid sequence in target cells in the sample to produce first recombinant target cells;
b. contacting the sample of one or more target cells resulting from step (a) with a first selection substrate, wherein the first recombinant target cells are selected if they express the first selection marker to produce a sample of first selected recombinant target cells;
c. contacting the sample of one or more target cells resulting from step (b) with a second exogenous nucleic acid sequence, and allowing the second nucleic acid sequence to exchange with a nucleic acid sequence in the one or more first selected recombinant target cells to produce one or more further recombinant target cells; and
d. contacting the sample of one or more target cells resulting from step (c) with a second selection substrate, wherein the one or more further recombinant target cells are selected if they lack expression of the second selection marker, thereby providing a sample of selected further recombinant target cells.

20. A method of in vivo serial alteration of two or more nucleic acid sequences of interest in a target cell comprising the steps of:
a. contacting a sample of one or more target cells with a first exogenous nucleic acid sequence having a first selection marker and a second exogenous nucleic acid sequence having a second selection marker, and allowing the first and second exogenous nucleic acid sequences to exchange with corresponding nucleic acid sequences in target cells in the sample to produce first recombinant target cells; and
b. contacting the sample of one or more target cells resulting from step (a) with a first selection substrate and a second selection substrate, wherein the first recombinant target cells are selected if they express the first selection marker and the second selection marker to produce a sample of first selected recombinant target cells.

21. A method of in vivo serial alteration of a nucleic acid sequence of interest in a target cell comprising the steps of:
a. contacting a sample of one or more target cells simultaneously or in series with one or more exogenous nucleic acid sequences having one or more selection markers, and allowing each exogenous nucleic acid sequence to exchange with a nucleic acid sequence in target cells in the sample to produce recombinant target cells;
b. contacting the sample of one or more target cells resulting from step (a) with one or more selection substrates, wherein the recombinant target cells are selected on the basis of positive or negative selection.

22. A method of in vivo serial alteration of a nucleic acid sequence of interest in a target cell comprising the steps of:
a. contacting a sample of one or more target cells with a first exogenous nucleic acid sequence having a selection marker, and allowing the first exogenous nucleic acid sequence to exchange with a nucleic acid sequence in target cells in the sample to produce first recombinant target cells and non-altered target cells;
b. contacting the sample of one or more target cells resulting from step (a) with a first selection substrate, wherein the recombinant target cells survive if they express the selection marker to produce a sample including surviving recombinant target cells and non-altered target cells die if they lack expression of the selection marker;
c. contacting the sample of one or more target cells resulting from step (b) with a second exogenous nucleic acid sequence, and allowing the second exogenous nucleic acid sequence to exchange with a nucleic acid sequence in the surviving recombinant target cells to produce a sample including further recombinant target cells and non-altered target cells; and
d. contacting the sample of one or more target cells resulting from step (c) with a second selection substrate, wherein the further recombinant target cells survive if they lack expression of the selection marker, and non-altered target cells die of they express the selection marker thereby providing a sample of living further recombinant target cells.
